(19) United States
(12) Patent Application Publication
(10) Pub. No.: US 2011/0270139 A1
(43) Pub. Date: Nov. 3, 2011

(54) SHOCK WAVE THERAPY APPARATUS FOR EXTRACORPORAL SHOCK-WAVE THERAPY

(75) Inventors: Edgar BAUER, Kraichtal (DE); Werner KRAUSS, Knittlingen (DE)

(73) Assignee: RICHARD WOLF GMBH, Knittlingen (DE)

(21) Appl. No.: 13/092,298

(22) Filed: Apr. 22, 2011

(30) Foreign Application Priority Data
Apr. 29, 2010 (DE) DE102010018707.0

(51) Int. Cl. A61B 17/225

(52) U.S. Cl. 601/4

(57) ABSTRACT

A shock wave therapy apparatus having a shock wave source and a control device for activating the shock wave source is provided for extracorporal shock wave therapy. The control device comprises a program module in which at least one predefined sequence of individual shock waves is stored or may be set. The shock wave source may be activated by the control device in a manner such that the shock wave source emits the predefined sequence of shock waves.

### BACKGROUND OF THE INVENTION

The invention relates to a shock wave therapy apparatus for extracorporal shock-wave therapy, having a shock wave source and a control device for activating the shock wave source.

Extracorporal shock wave therapy (ESWT) has been successfully applied for several years in orthopedics for non-invasive treatment of soft-part tissue which is close to the bone. Standard indications are for example tendinitis, tennis elbow and golfer elbow, patella point syndrome, tibia edge syndrome, archillodynia, and heel spur. Further indications are the so-called trigger points in muscles. Moreover, shock wave therapy is applied with more recent indications, for example wound treatment, cellulite, fat tissue or with chronic pelvic floor syndrome, wherein large-surfaced tissue areas are to be treated with these therapies.

With the therapies, shock waves are applied on one or more points of an area, wherein usually one starts with shock waves of a lower intensity and then the intensity in the course of the treatment can be increased, since the shock waves have a sedating effect. For this, continuous changes of the settings at the shock wave generator are necessary during the treatment.

### BRIEF SUMMARY OF THE INVENTION

It is the object of the invention to create an improved shock wave therapy apparatus with which, on the one hand, the course of treatment may be improved and, on the other hand, an improved adaptation of the course of treatment to the requirements of the individual patient, as well as to the respective indication, is possible.

This object is achieved by a shock wave therapy apparatus of the type mentioned at the outset, wherein the control device comprises a program module in which at least one predefined sequence of individual shock waves is stored or may be set, and the shock wave source may be activated by the control device in a manner such that the shock wave source emits the predefined sequence of shock waves. Preferred embodiments are to be deduced from the subsequent description as well as the attached figures.

The shock wave therapy apparatus according to the invention, for extracorporal shock wave therapy, as with known apparatus, comprises a shock wave source and a control device for activating the shock wave source. For example, the individual shock waves are triggered and the intensity is set by the control device.

According to the invention, the control device is provided with a program module which permits further presettings. Thus, the program module is envisaged for producing predefined sequences of shock waves. That is, the control device activates the shock wave source in a manner such that it automatically emits a predefined shock wave sequence consisting of a multitude of individual shock waves. The predefined sequence or succession of shock waves is stored in the program module or may be present at this module. On triggering or activating the shock wave source, the control device then controls the shock wave source, such that this emits the predefined sequence of shock waves. The treatment may be significantly simplified in this manner and may also be better adapted to the individual requirements of the respective indication or of the respective patient. Thus for example, it is possible to store or set predetermined, defined sequences of individual shock waves for certain forms of therapy or also for certain patients, in the program module. During the treatment then, a continuous change of the settings at the therapy apparatus is not necessary, since for example, changes of intensity and frequency are preset via the program module, so that the control device then automatically produces the predefined shock wave sequence via the program module.

Preferably, the control device is designed such that at least one predefined sequence of individual shock waves of a different intensity is stored or may be set on the program module. This allows a sequence of individual shock waves which have different intensities, to be preset or defined at the program module. Thus, it is possible, for example, to increase the intensity in the sequence in an automated manner, so that the sedating effect of the shock waves is utilized, in order to be able to apply shock waves of a greater intensity in a manner which is as pain-free as possible. The actual treatment is then simplified by the possibility of presetting the intensity courses in a sequence of a multitude of individual shock waves via the program module, since settings of the intensity no longer need to be carried out in a manual manner during the treatment.

Alternatively or additionally, the program module may be designed such that at least a predefined sequence of individual shock waves of a different frequency, of impulse duration and/or with differently long pauses, is stored or may be set. Thus for example, the total impulse duration of a multitude of consecutive individual shock waves may be preset. In a sequence of such shock waves, moreover, the length and the number of pauses between the individual impulses of individual shock waves may be preset and in particular varied in a predefined manner over the complete sequence of individual shock waves. The pauses serve for recovery or regeneration, in order to prevent an overloading of the tissue. Thus for example, after a certain number of shock waves, one may envisage a somewhat longer pause, before a further number of shock waves is applied in an automatic manner again. The number of individual shock waves which are emitted per unit of time may be varied by changing the frequency. Preferably, the formation of longer total impulses and pauses lying therebetween is effected by an amplitude modulation of a basic base frequency of individual shock waves.

The control device is, moreover, preferably designed for the production of different intensity courses in the sequence of individual shock waves. This may be effected in the previously described manner, by consecutive individual shock waves which are emitted with a predefined frequency, being changed in their amplitude, i.e. intensity, such that total impulses composed of the individual impulses are formed with a changing intensity. Thus for example, a sinusoidal, sawtooth-shaped or rectangular impulse course may be realized over the complete sequence of the shock waves. The intensity course of the sequence of shock waves may also be preferably stored or preset via the program module. Thereby, one may also store or preset variations of the intensity course over the whole sequence.

The program module is preferably provided with a memory, in which several predefined sequences of shock waves are stored or may be stored. Thereby, these sequences of individual shock waves may have the previously described variations in intensity, frequency, impulse duration and/or pause duration. Such a memory permits the storage of sequences which are envisaged for different applications or indications. Thus, the user may then select a predefined sequence of shock waves for the respective indication, at the program module, and apply these. Alternatively or additionally, the program module may be designed such that sequences of shock waves, which have been set once, may be individually stored for later treatments. A storage of sequences of shock waves, which are set or selected in a patient-specific manner, is also conceivable. In this manner then, with the treatment, the setting of the therapy apparatus is significantly simplified, since, of a number of predefined or previously stored sequences or series of shock waves, one only needs to select the desired ones in each case and then activate after placing the therapy head or the shock wave source. Further settings of the therapy apparatus are then preferably no longer necessary.

It is further preferable for the program module to be designed such that at least one sequence of individual shock waves is stored or may be set, whose frequency, intensity, pause duration and/or impulse duration vary in a cyclical manner. Thus, it is possible for example to preset sequences of shock waves, which in a cyclical manner increase the intensity of the shock waves and after a number of individual impulses envisage a certain pause or time duration with shock waves of a lower intensity, which is envisaged as a recuperation phase for the tissue.

Usefully, at least one input device is provided on the shock wave therapy apparatus, by which a predefined sequence of shock waves may be selected or set at the program module. The input device may, for example, be designed in the form of input keys, rotary dial or slider control, a computer mouse, a touch pad or in another known manner. The user, via the input device, may either select stored sequences of shock waves at the program module or, as the case may be, also set and store new sequences at the program module or also vary or change previously set or stored sequences. The control device is designed accordingly, in order to permit corresponding settings via the input device.

Particularly preferably, the input device comprises a touch-sensitive screen on which, for example, the desired settings may be carried out, for example by an input pen, or one may carry out the desired setting with the fingers. A very simple operation is thereby made possible, and the number of operating elements is simultaneously reduced.

Particularly preferably, the control device here is designed in a manner such that the course of the curve of a sequence of shock waves may be preset by moving a curve on the screen. This may be effected, for example, by certain points of the curve being marked in a manual manner and then displaced such that a desired curve course results. The marking and displacement may, for example, be effected via an input element such as a computer mouse or with the use of a touch-sensitive screen, also in a direct manner on the screen by an input pen or ideally directly with the fingers. In this manner, it is very simple to preset a sequence of shock waves with a desired intensity course and, as the case may be, with pauses, at the program module.

According to a further preferred embodiment, the control device has a learn mode, in which a treatment sequence with a special sequence of individual shock waves may be learned and stored in the program module. Thus, the learn mode may be designed such that first a sequence of individual shock waves may be manually set and then stored in the program module as a predefined shock wave sequence.

The shock wave therapy apparatus according to the invention, as has been previously described, permits a new type of therapy method by shock waves. With this therapy method, in an automated manner, one may apply sequences of predefined individual shock waves onto the tissue. The shock wave source or the therapy head may be placed and directed at the desired location of the body for this. Subsequently, a previously selected, predefined shock wave sequence is activated and then applied from the shock wave source onto the tissue in an automated manner, without yet further manual settings and in particular changes of the parameters being necessary during the application of the shock wave sequence with this application. A thus applied predefined shock wave sequence is preferably designed such that the intensity of the shock waves is varied in the course of the shock wave sequence, preferably first in a stepped manner or linearly increased up to a maximum and subsequently is reduced in a sudden manner, in steps or linearly, so that shock waves of a greater intensity alternate with pauses, in which no or only shock waves with a lower intensity are applied during the treatment. The pauses form recuperation phases for the tissue. Thereby, different intensity courses, for example with rectangular, sawtooth-shaped or wave-like courses may be applied. The length of the pauses in the total course may also be varied. Thus in each case, a longer pause may be envisaged, for example after several longer impulses which, as the case may be, are formed from a multitude of individual impulses, in order to permit a recuperation of the tissue. Further aspects of the method are to be deduced from the above description of the therapy apparatus.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a schematic diagram of the construction of a shock wave therapy apparatus according to an embodiment of the invention;

FIGS. 2a-2d are intensity curves over time of four possible intensity courses of a shock wave sequence;

FIG. 3a is an intensity curve of a shock wave sequence according to a first variation;

FIG. 3b is an enlarged detail of the portion B of the curve in FIG. 3a;

FIG. 4a is an intensity curve of a second variant of a shock wave sequence;

FIG. 4b is an enlarged detail of the portion B of the curve in FIG. 4a;

FIG. 5a is an intensity curve of a third variant of a shock wave sequence;

FIG. 5b is an enlarged detail of the portion 1 in the curve of FIG. 5a;

FIG. 5c is an enlarged detail of the portion 2 in the curve of FIG. 5a; and

FIG. 6 is a schematic diagram showing the change of the course of the curve of the intensity of a shock wave sequence.

### DETAILED DESCRIPTION OF THE INVENTION

The shock wave therapy apparatus shown in FIG. 1 comprises a control device 2 and a therapy head or a shock wave source 4, which is connected via a cable 6 to the control device 2. A button 8 for triggering or activating shock waves or shock wave sequences is attached on the shock wave source 4.

The control device 2 comprises, as essential components, an input device 10, a screen 12 and an HV-unit 14, which produces the high-voltage impulses for the shock wave source 4. A central processing unit (CPU) 16, which is connected to the screen 12, the input device 10 and the HV unit 14 for signal or information exchange, is provided in the control device 2. According to an embodiment of the invention, a program module 18 is further present in the control device 2. This may be designed as its own hardware or only as a software module, which runs in the control device 2, in particular the program module 18.

Whereas with conventional shock wave therapy apparatus, it was only the intensity and impulse duration of the individual shock wave impulse which could be preset at the control device 2, and which was then emitted by the shock wave source 4 on activation via the button 8, it is possible via the program module 18 envisaged according to the invention, to define predefined sequences or series of a multitude of individual shock waves prior to this in the control device 2. Thereby, the intensity, frequency, impulse duration, and/or possible pauses between individual shock waves may vary in this sequence. Such shock wave sequences may be stored in a predefined manner in the program module 18 for different indications, so that a predefined shock wave sequence may be selected via the input device 10 and the screen 12 from the memory of the program module 18 depending on the application purpose. It may also be possible to store in the program module 18 individual, patient-specific shock wave sequences for later treatments, and to select them accordingly. For this, one may either vary or change predefined shock wave sequences via the input device 10 and the screen 12, and subsequently store them, or however, as the case may be, even program new shock wave sequences and subsequently store them in the program module 18 for later applications.

After the selection of a predefined shock wave sequence or setting of a predefined shock wave sequence at the program module 18, then it is only the emitting of this shock wave sequence which is started by actuating the button 8. Subsequently, the control device 2, in particular its central control unit 16, on the basis of the information from the program module 18, then activates the HV-unit 14 such that the shock wave source 4 emits the shock waves according to the predefined shock wave sequence, without yet further adaptations or changes being necessary for this during the application. That is, according to the invention, one envisages a shock wave sequence either being previously stored in the program module 18 and selected, or however being preset in the program module 18 before the application and then being completely worked away by the control device 2 on activation via the button 8, so that the complete sequence is then produced in an automated manner by the shock wave source 4. That is, the predefined individual shock waves are automatically emitted successively by the shock wave source 4, according to the predetermined respective frequency, intensity, pulse duration and, as the case may be, pause length between the individual shock waves. Due to this method, it is significantly simpler to apply comprehensive shock wave variations during the treatment, in particular to vary the intensity and pause duration such that the intensity may be increased in a stepped manner, in order to prevent pains and, as the case may be, to provide sufficient pauses as recuperation phases for the tissue. The user no longer needs to carry out further adaptations during the application, in order to achieve this.

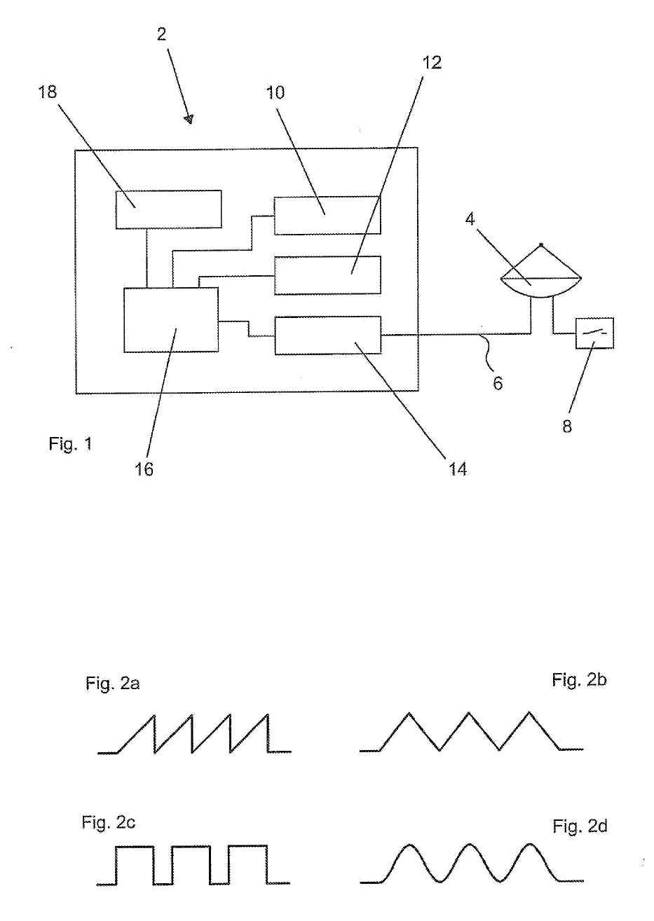

It is particularly possible, to vary the intensity course of the shock waves over time in different shapes. Here, it is preferable for the intensity to increase in a cyclical manner and then drop again. Due to the increase, one succeeds in being able to increase the intensity in an as painless manner as possible, while utilizing the sedating effect of the shock waves, and on the other hand, by the cyclical reduction of the intensity which is effected again and again, one succeeds in providing the tissue with adequate regeneration phases between the individual shock wave pulses. FIG. 2a shows a sawtooth-shaped intensity course over time. FIG. 2b shows a zigzag course, while FIG. 2c shows a rectangular course of the intensity curve and FIG. 2d a wave-like intensity course over time. It is to be understood that one may also apply other variations of the intensity over time, in particular also combinations of the shown intensity courses.

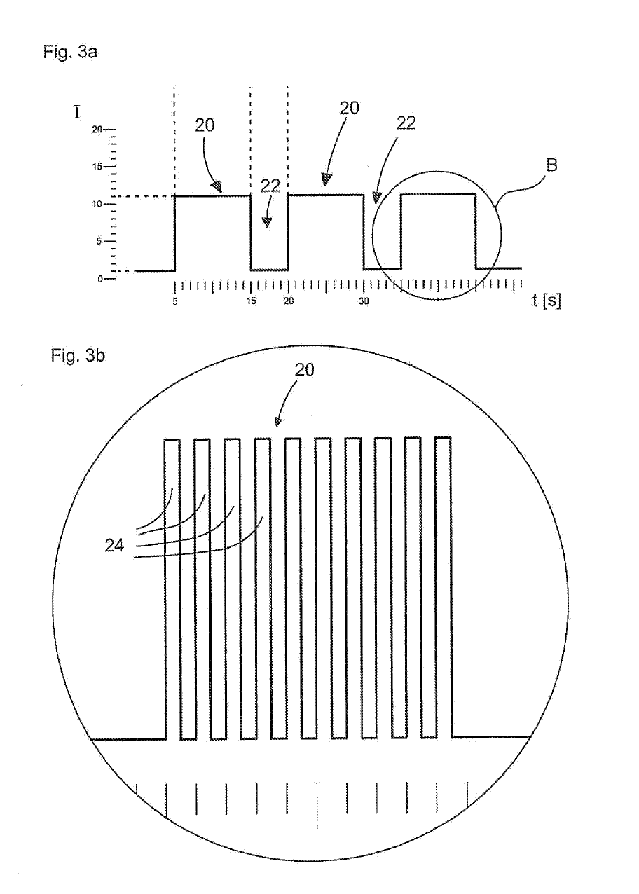

One example of a rectangular intensity course is explained in more detail by FIGS. 3a and 3b. In FIG. 3a the intensity change is effected in a sudden manner between the intensity stages 1 and 11, whereby a rectangular intensity course of the shock wave sequence is achieved. The rectangular intensity course of the shock wave sequence is realized as follows with the embodiment example. The basis is a shock wave sequence with a constant frequency of one Hertz. This means that in each case, a shock wave is emitted in intervals of one second. The intensity of the shock waves is varied between two intensity steps, specifically the steps 1 and 11. First, shock waves with the intensity step 1 are emitted up to the point in time of five seconds. At the point in time of five seconds, the intensity is increased in a sudden manner to the intensity step 11. Subsequently, as shown in FIG. 3b, ten individual shock waves with the greater intensity are emitted, whereby a shock wave impulse 20 is formed. Subsequently, after ten seconds, which is to say at the point in time of fifteen seconds shown in FIG. 3a, the intensity is reduced again to the intensity step 1, and this is retained with a constant shock wave frequency up to the point in time of twenty seconds. Thus, a pause 22 with a lower intensity of the shock waves is created, which serves for the regeneration of the tissue. Subsequently, the intensity is increased again and held for tens seconds at a shock wave frequency of one Hertz, so that a second impulse 20 is formed, and so on. The intensity course shown in FIG. 3a, having impulses 20 separated by the pauses 22, is thus formed by amplitude modulation of a basic sequence of individual shock waves 24 having a constant frequency.

Such a shock wave sequence, as is shown in FIG. 3a, may be preset in the program module 18, so that then, on actuation of the button 8, such a shock wave sequence is emitted by the shock wave source 4. Here, the control device 2 may control the shock wave sequence such that it emits these for a defined time duration, which as the case may be, may likewise be preset in the program module 18. Alternatively, the shock wave sequence may also be finished in a manual manner, for example by once again actuating the button 8.

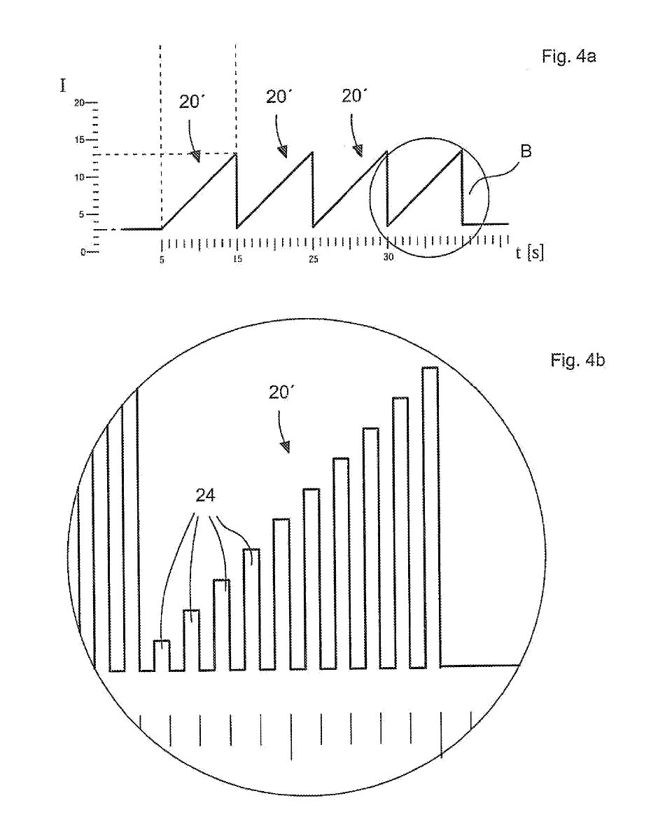

FIGS. 4a and 4b, similarly to the FIGS. 3a and 3b, show a further example of a shock wave sequence. This shock wave sequence has a sawtooth-shaped curve course corresponding to the curve course in FIG. 2a. Here, a multitude of sawtooth-shaped impulses 20′ connect directly to one another without pauses. The individual impulses 20′ are also produced by a plurality of individual shock waves 24, which are emitted with a fixed frequency, here 1 Hertz. With the example according to FIGS. 4a and 4b, the intensity of the individual shock waves 24 does not however only change between two intensity values, but increases from individual shock wave 24 to individual shock wave 24 in ten steps, as seen in FIG. 4b. The slow rise of the intensity in the impulse 20′ results by this. The intensity is then suddenly reduced to the lowest intensity at the end of the impulse, to step 3 in the shown example. Subsequently, the intensity increases again in 10 steps, over a time period of 10 seconds, to the maximum intensity of step 13.

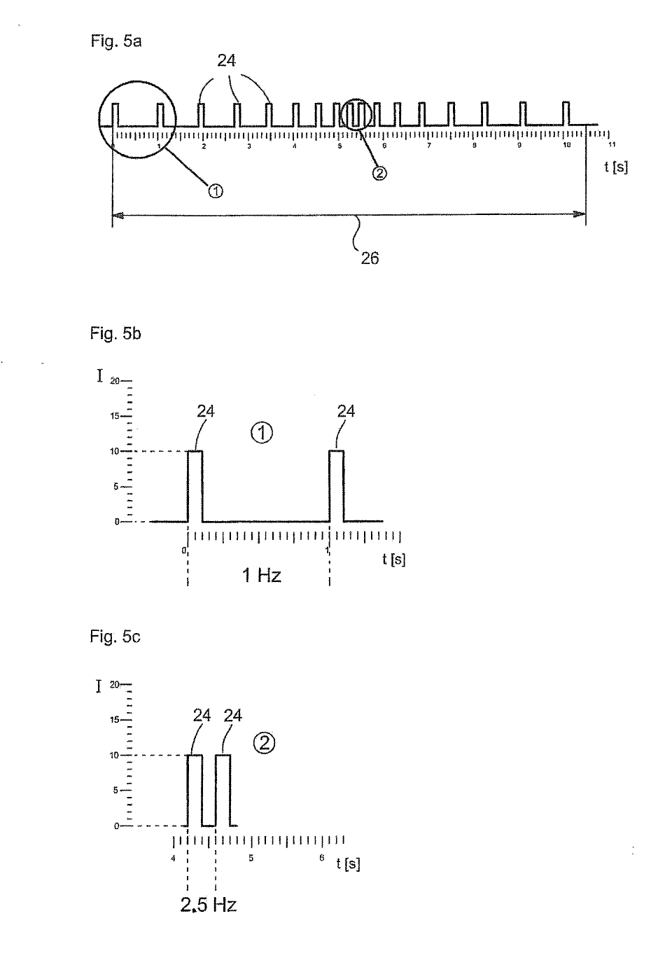

The shock waves sequences in the examples of FIGS. 3 and 4 were based on a fixed basic frequency for shock waves 24. With the treatment, the tissue of the organism may react to the uniformly applied shock waves in a manner such that a habituation effect sets in. A wobble function may be envisaged to counteract this, in a manner such that the frequency is cyclically varied, which is explained by FIGS. 5a to 5c. In these Figures too, the intensity I is plotted over time t in seconds. The frequency of the individual shock waves 24 is varied over a time interval 26, in this example of 9 seconds. First, one begins with a low frequency of 1 Hertz (see detail 1 in FIG. 5b). This frequency is subsequently increased to a maximum of 2.5 Hertz, which is shown in the detail 2 in FIG. 5c, and subsequently the frequency falls again to the frequency of 1 Hertz. Thus, first an increase of the frequency and then a drop occur. This is repeated in a cyclical manner with an interval duration 26 of 9 seconds in this example. Such a frequency change may be preset at the program module 18 or stored in the program module 18. Then, after a selection of such a shock wave impulse, merely an activation via a button 8 is effected, so that this shock wave sequence is then emitted by the shock wave source 4 for a defined time or up to a renewed activation of the button 8. In the example according to FIG. 5a to FIG. 5c, the intensity of the shock waves is constant. However, it is to be understood that here too, a variation of the intensity could be effected, as has been explained for example by FIGS. 3 and 4. Thus, it is also possible to combine frequency change and a change of the pause durations with an intensity change.

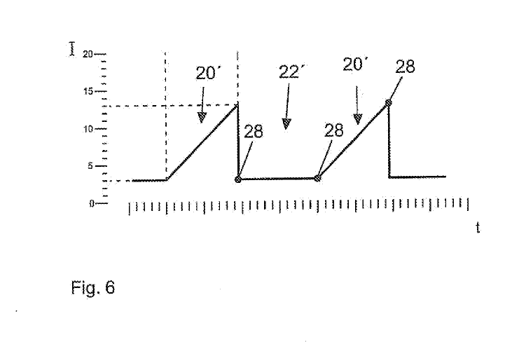

With this embodiment, one envisages the curve being represented on the screen 12 and the curve being able to be changed directly on the screen 12, in order to be able to simply select or preset the course of the curve of the shock wave sequence. This may be effected by the touch-sensitive design of the screen, so that individual marking points 28 (FIG. 6) on the curve may be displaced into the desired shape directly on the screen with a pen or with the finger. Alternatively, a displacement may also be effected by a mouse dial and by a separate input device 10. In FIG. 6 a curve course, for example, is shown similarly to that in FIG. 4a, with the difference that here, in each case, a pause 22′ is provided between two impulses 20′. By displacing the marking points 28 the curve, on the one hand, may be brought into a different shape and, on the other hand, the duration of the impulses 20 and the duration of the pauses 22′ may be changed in a simple manner. The intensity may also be easily changed by displacing the points 28. Thus, a very simple operation of the shock wave apparatus according to the invention and in particular a very simple presetting of a defined shock wave sequence are possible.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A shock wave therapy apparatus for extracorporal shock wave therapy, comprising a shock wave source (4) and a control device (2) for activating the shock wave source (4), the control device (2) comprising a program module (18) in which at least one predefined sequence of individual shock waves (24) is stored or may be set, wherein the shock wave source (4) is activatable by the control device (2) in a manner such that the shock wave source (4) emits the predefined sequence of individual shock waves (24).

2. The shock wave therapy apparatus according to claim 1, wherein at least one predefined sequence of individual shock waves (24) of a different intensity (I) is stored or may be set in the program module (18).

3. The shock wave therapy apparatus according to claim 1, wherein at least one predefined sequence of individual shock waves (24) of a different frequency, impulse duration (20) and/or with differently long pauses (22) is stored or may be set in the program module (18).

4. The shock wave therapy apparatus according to claim 1, wherein the control device (2) is designed for producing different intensity courses in the sequence of individual shock waves (24).

5. The shock wave therapy apparatus according to claim 1, wherein the program module (18) is equipped with a memory in which a plurality of predefined sequences of shock waves (24) are stored and/or storable.

6. The shock wave therapy apparatus according to claim 1, wherein at least one sequence of individual shock waves (24) is stored or may be set in the program module (18), and wherein the frequency, intensity (I), pauses (22) and/or impulse duration (20) of the sequence of shock waves vary cyclically.

7. The shock wave therapy apparatus according to claim 1, further comprising at least one input device (10) by which a predefined sequence of shock waves (24) is selectable and or set at the program module (18).

8. The shock wave therapy apparatus according to claim 7, wherein the input device (10) comprises a touch-sensitive screen (12).

9. The shock wave therapy apparatus according to claim 7, wherein the control device (2) is designed in a manner such that a curve course of a sequence of shock waves may be preset by moving a curve on the screen (12).

10. The shock wave therapy apparatus according to claim 1, wherein the control device (2) comprises a learning mode, in which a treatment sequence having a special sequence of individual shock waves (24) may be learned and stored in the program module (18).
